(19) United States
(12) Patent Application Publication
(10) Pub. No.: US 2019/0298242 A1
(43) Pub. Date: Oct. 3, 2019

(54) COMMUNICATION SYSTEM AND WEARABLE APPLIANCE

(71) Applicant: UNIVERSITE DU LUXEMBOURG, Luxembourg (LU)

(72) Inventor: Manuela Jungmann, Trier (DE)

(21) Appl. No.: 16/331,331

(22) PCT Filed: Sep. 6, 2017
(86) PCT No.: PCT/EP2017/072371

§ 371 Date: Mar. 7, 2019

(30) Foreign Application Priority Data
Sep. 7, 2016 (LU) 93199

(51) Int. Cl. A61B 5/16; A61B 5/00; G06F 3/01

(52) U.S. Cl. CPC A61B 5/165; A61B 5/0022; A61F 2007/0093; G06F 3/016; G06F 3/015

(57) ABSTRACT

A wearable appliance or a set of wearable appliances, arranged for being worn by a person on the person's body comprises: one or more cold applicators, one or more biosignal sensors for measuring biosignals indicative of physiological stress experienced by the person and control circuitry connected to the one or more cold applicators and configured so as to activate them in response to detection, based upon the measured biosignals, that the person experiences elevated physiological stress. According to a further aspect, the invention proposes a communication system comprising a wearable appliance (24) or a set of wearable appliances (24, 26) and one or more mobile terminals (22) comprising each a human interface device with one or more output devices (40, 46, 50) for indicating the level of stress to a second person holding the mobile terminal and with one or more input devices (44, 50) for receiving input from that person. The system translates input received by the one or more mobile terminals into a control signal provided to the one or more cold applicators (18).

### FIELD OF THE INVENTION

The invention generally relates to a communication system, in particular, for enabling non-verbal communication between humans over a distance. An aspect of the invention relates to a wearable appliance or a set of wearable appliances that can be used in such a communication system.

### BACKGROUND OF THE INVENTION

Fear of public speaking (also: glossophobia, speech anxiety) is widely spread and represents a significant problem for people experiencing it severely as it makes the achievement of their personal goals or career advancement more difficult, if not impossible. Several approaches for the treatment exist, e.g. cognitive behavioral therapy (CBT), medication, autogenic relaxation, etc., and are preferably applied in combination with dedicated training.

The present invention proposes a technological tool that can be used in the treatment of fear of public speaking or, more generally, in situations where a person is confronted with a challenge causing them to experience physiological stress. Other possible applications of the invention include non-invasive mitigation of acute stress, e.g. by stimulation of (parts of) the parasympathetic nervous system. For instance, in a certain aspect, the invention could be employed to give relief to people in stressful situations, e.g. to people suffering from a phobia (e.g. fear of flying, etc.) or to people in stressful situations such as working in a call center or as a trader in the stock market.

### SUMMARY OF THE INVENTION

A first aspect of the invention relates to a wearable appliance or a set of wearable appliances, arranged for being worn by a person (hereinafter also referred to as the wearer) on the person's body. The wearable appliance or the set comprises:

one or more cold applicators for applying cold to the person's body;
one or more biosignal sensors for measuring biosignals indicative of physiological stress experienced by the person; and
control circuitry connected to the one or more cold applicators and configured so as to activate the one or more cold applicators in response to detection, based upon the measured biosignals, that the person experiences elevated physiological stress.

As used herein, the term “biosignal” designates a signal in a living being that can be measured continuously, e.g. heart beat (or heart rate), respiration rate, electrodermal activity (galvanic skin response), etc. As is known in the art, these signals change if the subject experiences stress: the frequency of the heart beat signal and skin conductivity increase, respiration becomes flatter (amplitude of the respiration signal decreases) and respiration frequency increases. The signals may be used individually or in combination to detect the level of physiological stress. Preferably, the level of physiological stress is deduced from heart rate, e.g. using a threshold-based classifier. More preferably, the level of physiological stress is deduced from heart rate in combination with at least one of respiration rate and electrodermal activity, e.g. by using a classifier previously trained with data of the first person. The thresholds for detecting stress levels are preferably defined depending on predetermined individual-specific baselines, e.g. as a certain percentage above the heart rate, respiration rate or skin conductivity of the first person at rest or in another standardized situation.

The wearable appliance or set of wearable appliances allows the wearer to be automatically administered cold stimuli when they experience acute stress. The cold applicators are preferably configured and arranged on the wearable appliance(s) so as to stimulate (directly or indirectly) the wearer's parasympathetic nervous system through application of cold pulses. By stimulation of the parasympathetic nervous system (promoting maintenance of the body at rest), the sympathetic nervous system (responding to stress) is counteracted, and the level of stress subjectively experienced by the wearer may be reduced.

According to an embodiment, the wearable appliance or the set of wearable appliances comprises a monitor unit connected with the one or more biosignal sensors and configured for deducing the level of physiological stress from the biosignals and for monitoring the level of physiological stress or is configured for being connected (by cable or via a wireless link) with such a monitor unit. The control circuitry is preferably configured for controlling the one or more cold applicators based upon the level of physiological stress deduced by the monitor unit. The biosignal sensors, the monitor unit and the control circuitry may form a feedback loop automatically controlling the cold applicators based upon the level of physiological stress.

The control circuitry may e.g. comprise or consist of a microcontroller for driving the cold applicators. Preferably, the cold applicators are Peltier coolers that are activated by sending current pulses through them. Intensity and duration of the cold stimuli then depend directly on the amplitude and the duration of the current pulses, which are controlled by the microcontroller.

The one or more biosignal sensors could comprise a heart rate sensor and/or an electrodermal activity sensor.

According to an embodiment, one or more cold applicators are arranged in such a way as to be located on the wearer's neck, preferably in the inferior neck region where the neck connects to the torso, or on the superior portion of the torso where the torso connects to the neck, and more preferably in the lateral inferior neck region.

The wearable appliance or set of wearable appliances could comprise a neckwear item (any item worn on or around the neck, e.g., a necklace, a neckband, a collar integrated or not into a garment, such as, e.g., a necktie, a shirt, a blouse, etc.), wherein part of or all the cold applicators are located. Part of or all the cold applicators could be arranged within one or more patches or patch-like items that are attachable to the wearer's skin and/or to clothing and/or to accessories worn on the body. Such patches could be attachable to the wearer's skin using skin-friendly adhesive (e.g. skin-friendly pressure-sensitive adhesive) and/or to clothing or accessories using integrated clips or pins (e.g. safety pins) or any other suitable means.

Preferably, the wearable appliance or set of wearable appliances comprises a communication interface, most preferably a wireless communication interface (Bluetooth module, WiFi module, or the like), e.g. for communicating with a mobile device.

A second aspect of the invention relates to a communication system comprising:

a wearable appliance or a set of wearable appliances arranged for being worn by a first person (e.g. a speaker or a person in a potentially stressful situation) on the first person's body, preferably (directly) on the skin, the wearable appliance or the set comprising one or more cold applicators (e.g. Peltier coolers) for applying cold to the first person's body and one or more biosignal sensors for measuring biosignals indicative of physiological stress experienced by the first person;
a monitor unit configured (e.g. programmed) for monitoring the level of physiological stress experienced by the first person, the level of physiological stress being deduced from the measured biosignals; and
one or more mobile terminals comprising each a human interface device with one or more output devices for indicating the level of physiological stress to a second person (e.g. a person sitting in the audience or a friend of the first person) holding the mobile terminal and with one or more input devices for receiving input from the second person.
The communication system is configured to translate input received by the one or more mobile terminals from one or more second persons into a control signal provided to the one or more cold applicators.

The communication system preferably includes a wearable appliance or a set of wearable appliances as presented hereinabove. As will be appreciated, the communication system allows the one or more second persons to activate the one or more cold applicators and thereby provide the first person with a cold stimulus in case they notice that the first person is experiencing stress. The level of physiological stress experienced by the first person is communicated to the one or more mobile terminals in real time (i.e. nearly instantaneously). Likewise, the translation of the inputs received by the one or more mobile terminals into a control signal provided to the one or more cold applicators also happens in real time.

The communication system can be used for collaborative interaction in any situation involving a person facing a challenge. For instance, the communication system can be used to transmit one or more audience members' “social touch” remotely to the person speaking. The audience members allowed to interact with the speaker via the communication system (audience participants) are selected before the speech. They can be friends of the speaker or unknown to him. They are instructed to look out for the speaker showing signs of anxiety and to send him a “social touch” (reassuring stimulus) when they feel that it is necessary. The audience participants have different sources of information on which to base their decision as to whether the speaker becomes increasingly nervous. One source of information is the feedback delivered by the one or more output devices of the mobile terminals. Another source of information is the visual appraisal of the speaker's demeanour during the talk. The “social touch” is conveyed as a cold stimulus, which tells the speaker that the audience members support him or her. Furthermore, application of cold stimulus may facilitate relaxation and gaining mental calmness. For the audience participants, the application enhances the participants' self-awareness of empathy through active identification with the speaker's situation.

Preferably, the wearable appliance or the set of wearable appliances is configured such that it can be concealed under normal street clothing. For instance, the wearable appliance or at least one of the set of wearable appliances could be integrated into a vest, a formfitting undergarment (e.g. an undershirt or a bra), a harness, a neckwear item or a halter with straps for being worn on the torso. As used herein, the term “harness” designates an arrangement of straps for fastening something to a person's torso, e.g. a small backpack or pocket with shoulder straps for being worn on the torso.

Preferably, some or all of the one or more cold applicators are arranged in the vest, formfitting undergarment, harness, neckwear item or halter. The one or more cold applicators could be arranged on the inner surface of the vest, formfitting undergarment, harness, neckwear item or halter in such a way as to be located on the first person's back or on the first person's neck, preferably in the inferior neck region where the neck connects to the torso, or on the superior portion of the torso where the torso connects to the neck, and more preferably in the lateral inferior neck region, when the first person wears the vest, formfitting undergarment, harness, neckwear item or halter. The one or more cold applicators could be at least two cold applicators arranged in such a way as to be located symmetrically on either side of the first person's spine or neck.

Alternatively or additionally, some or all of the one or more cold applicators could be arranged on an armlet or a pair thereof so as to be applied on the first person's upper arm.

If the communication system comprises a set of wearable appliances (i.e. at least two), at least one of the set of wearable appliances could be integrated into a wristband or a chest belt. In this case, the at least one wearable appliance integrated into the wristband or chest belt preferably comprises at least one of the one or more biosignal sensors. Alternatively or additionally, at least one of the set of wearable appliances could be integrated into a neckwear item or a patch attachable to the first person's skin and/or to clothing and/or to accessories worn on the body.

The monitor unit could be any type of hardware capable of monitoring the level of physiological stress based on the measured biosignals. For instance, the monitor unit could comprise or consist of an application-specific integrated circuit (ASIC), a system on a chip (SoC), a programmable logic device (PLD), an erasable programmable logic device (EPLD), a programmable logic array (PLA), a field-programmable gate array (FPGA), a generic microprocessor and/or other hardware adequately programmed for the task, or the like. The monitor unit could run an artificial intelligence software configured to learn the wearer's stress pattern and to make adjustments (e.g. to detection thresholds, etc.) accordingly. Alternatively or additionally, the settings of the monitoring software could be fixed by the manufacturer and/or by the user, e.g. during a training phase.

The monitor unit could be integrated into the wearable appliance or into one of the set of wearable appliances. The monitor unit could alternatively be integrated into a mobile terminal of the one or more mobile terminals. As a further possibility, the monitor unit could be separate both from the wearable appliance or the set of wearable appliances and from the one or more mobile terminals. For instance, the monitor unit could be implemented on a remote computer connected to the wearable appliance or the set of wearable appliances and to the one or more mobile terminals by a wireless network and/or over the Internet.

According to an embodiment, the one or more biosignal sensors comprise a heart rate sensor and/or an electrodermal activity sensor. In this case, the monitor unit is preferably configured for monitoring the level of physiological stress as deduced from heart rate and/or electrodermal activity (e.g. galvanic skin response). Additionally or alternatively, the one or more biosignal sensors could comprise a breath rate sensor. The sensors are preferably positioned on the first person's body in such a way that the biosignals can be captured with sufficient accuracy. For instance, a breath rate sensor or a heart rate sensor would preferably be arranged on a chest belt. A wristband could be used for carrying a heart rate sensor and/or an electrodermal activity sensor. Electrodermal activity could, however, also be measured on a finger or the foot. A neckwear item may be particularly suitable for housing a heart rate sensor.

According to an embodiment, the one or more cold applicators are at least two cold applicators and the one or more mobile terminals are at least two mobile terminals, the mobile terminals being configured to send control signals to different cold applicators. In other words, each mobile terminal is in this case associated with a specific group of cold applicators and may control only that group. Each group of cold applicators may comprise one or more cold applicators. The groups controlled by the different mobile terminals are distinct, preferably disjoint. The association between mobile terminals and cold applicators may be static. Alternatively, it may change over time.

The communication system is preferably configured in such a way that the wearable appliance or the set of wearable appliances, the monitor unit and the one or more mobile terminals are configured to communicate by a wireless network, e.g. based on Bluetooth, WiFi (IEEE 802.11), ZigBee, etc., communication protocols.

According to an embodiment, the one or more output devices of the mobile terminals include electromechanical actuators for haptically indicating the level of physiological stress. As an electromechanical actuator may be used, for instance, an eccentric rotating mass (ERM) motor, a linear resonant actuator (LRA) or a piezo actuator. Instead or in addition to electromechanical actuators, the one or more output devices could comprise a visual output device, e.g. a display screen, an indicator lamp (e.g. integrated into a button used as an input device), etc.

### BRIEF DESCRIPTION OF THE DRAWINGS

By way of example, preferred, non-limiting embodiments and applications of the invention will now be described in detail with reference to the accompanying drawings, in which:

FIG. 1: is an illustration of a communication system according to an embodiment of the invention in use during a speech of a person in front of an audience;

FIG. 2: is a schematic illustration of how the components of the communication system of FIG. 1 cooperate to alleviate the speaker's stress;

FIG. 3: is a schematic view of a first form of a mobile terminal usable in the context of the invention;

FIG. 4: is a schematic view of a variant of the mobile terminal of FIG. 3;

FIG. 5: is a schematic view of a smart phone configured as a terminal usable in the context of the invention by an app;

FIG. 6: is a schematic illustration of how the components of the communication system of FIG. 1 may cooperate to alleviate the speaker's stress without manual intervention of another person;

FIG. 7: is a schematic illustration of a wearable appliance configured as a neckwear item;

FIG. 8: is a schematic illustration of a wearable appliance configured as a patch;

FIG. 9: is a schematic illustration of a wearable appliance configured as a patch combined with headphones;

FIG. 10: is an illustration of a preferred location for applying cold pulses.

### DETAILED DESCRIPTION OF PREFERRED EMBODIMENTS

As indicated above, a preferred application of the communication system of the present invention relates to the treatment of fear of public speaking. The preferred embodiments described herein are presented in such a context for illustration. It will be appreciated, however, that the invention may have other applications.

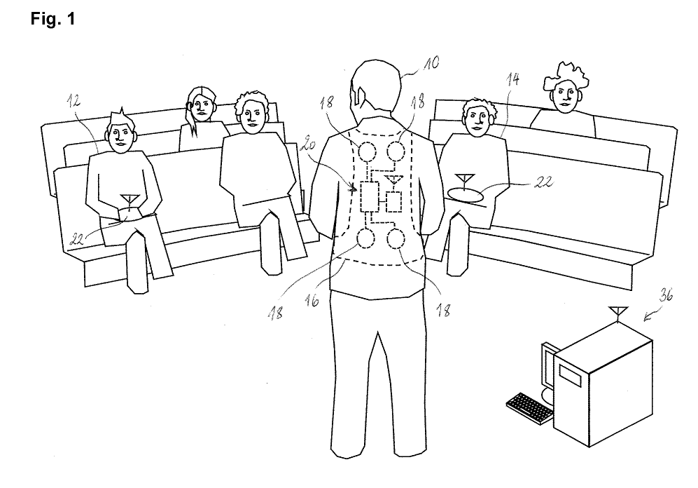

FIG. 1 is an illustration of a person (hereinafter: speaker) 10 in a public speech situation in front of an audience. A communication system according to an embodiment of the invention is used to transmit “social touches” of previously selected audience members 12, 14 remotely to the back of the speech-anxious speaker 10. The aim is to thereby alleviate the speaker's social anxiety during their talk. The speaker 10 wears a concealed light-weight halter 16, with built-in cold applicators 18 and control circuitry 20 for remotely controlling the cold applicators 18. The cold applicators 18 (preferably Peltier coolers) are oriented with their cold sides turned towards the speaker's back, in such a way that they administer a coldness stimulus when activated. The “social touch” are experienced by the speaker 10 as localised cooling sensations.

In the illustration, the halter 16 comprises two pairs of cold applicators 18 symmetrically placed each side of—and close to—the thoracic vertebral midline of the back. Alternative or additional positions for the cold applicators would be the on the upper arms. Generally speaking, the cold applicators are preferably placed in cold-sensitive areas of the human body rather than in cold-insensitive areas. Cold/hot-sensitive zones of the human body are known e.g. from human thermoregulation research. Cold-sensitive areas can be found, for instance in the lower back region left and right of the spine. Cold-sensitive areas coinciding with acupuncture positions that facilitate relaxation and mental calmness may be especially preferred for application of the cold stimuli.

The communication system provides a collaborative interface for the speaker and certain audience members 12, 14. As shown in FIG. 1, audience members 12, 14 hold each a mobile terminal 22. The mobile terminals 22 are conceived as human interface devices and comprise each at least one output device for indicating the speaker's level of physiological stress and at least one input device for receiving inputs from the respective audience member. The communication system translates the inputs received by the mobile terminals 22 into control signals for the cold applicators.

The audience members 12, 14 holding the mobile terminals 22 can activate the cold applicators at any time during the talk when they believe the speaker's anxiety level is high. To assess the speaker's anxiety level, they can rely not only on the indications provided by the mobile terminals but also on what they see and hear from the speaker 10. The audience members with the mobile terminals 22 may be friends of the speaker or persons the speaker has trust in (e.g. personal trainer, coach or therapist). The mobile terminals 22 may also be given to persons picked from the audience and instructed to send the speaker comforting signals (“social touches”) whenever they think the speaker is anxious or becomes increasingly nervous. When the communication system is used in this way, it requires from the audience participants to discern or, at least, attempt to discern the speaker's feelings and thereby promotes audience participants' empathy towards the speaker.

It will be appreciated that the approach presented herein is significantly different from conventional therapeutic intervention to assist speech-anxious individuals. Conventional approaches are often based on CBT programs. In recent years, virtual reality (VR) technology has been used to support CBT during exposure sessions. Aiming to desensitize the speaker, virtual audiences are simulated but focus on antagonistic audience presentation.

The present methodology inverses this approach in at least two ways:
i. A new perspective on speaker-audience relations is provided: audience members who operate the mobile terminals give reassurance feedback to the speaker, implying a positive affect, which is contrary to the negative affect that is used in exposure therapy.
ii. The present methodology uses a physiological approach rather than conventional cognitive approaches. The speaker is enabled to modify the mental pattern through a physical trigger (cooling sensation), whereas in CBT and other therapies speech anxiety is dealt with solely through mental means.

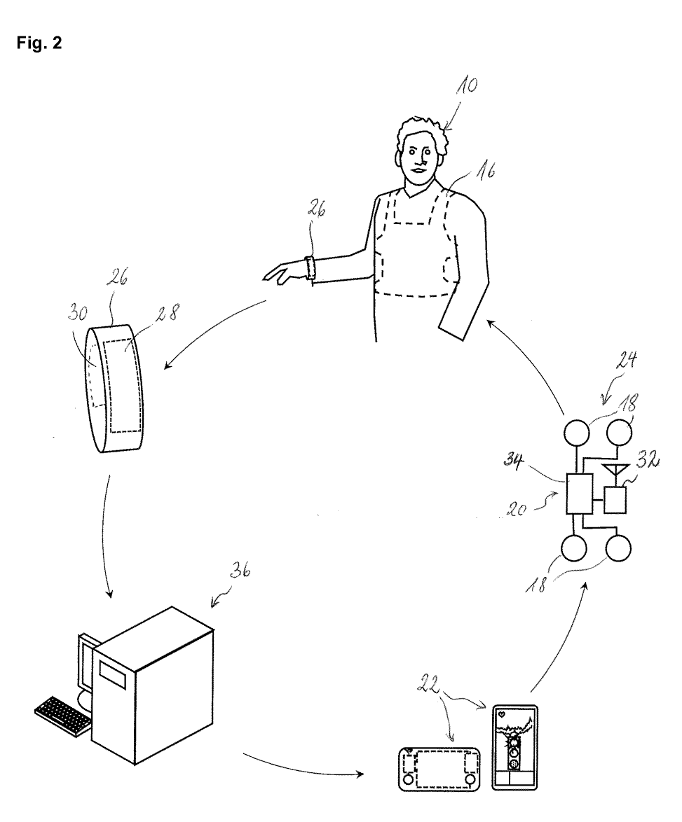

FIG. 2 schematically illustrates how the components of the communication system of FIG. 1 cooperate. The speaker 10 wears a halter 16 around his torso that comprises a first appliance 24 including the cold applicators 18 and the corresponding control circuitry 20. The speaker 10 also wears a second appliance 26 in the form of a wristband, which carries biosignal sensors 28, 30.

The control circuitry of the first appliance 24 comprises a microcontroller 32 with an embedded RF module and an amplifier circuit 34 connected to the cold applicators 18 and controlled by the microcontroller 32.

Turning to the wristband 26, the biosignal sensors include a heart rate sensor 28 and an electrodermal activity sensor 30. The wristband also includes an RF (radio frequency) module (not shown) for wireless communication with the other components of the system.

The biosignals measured by the wristband 26 are forwarded to a monitor unit, represented here by a general-purpose computer 36, which is also equipped with an RF module (not shown) and programmed to derive an indicator (level) of physiological stress experienced by the speaker from the biosignals and to monitor the level. Monitoring may comprise, for instance, comparing the level of physiological stress with one or more thresholds and classifying the stress level accordingly, e.g., as “calm”, “slightly nervous”, “nervous”, “anxious” and “panicking”, or the like, based on heart rate and electrodermal activity. Alternatively or additionally, monitoring may comprise one or more of the following: recording the deduced stress level over time, preparing a time series for visualization, computing statistics, calculating a current trend (stress prediction), etc.

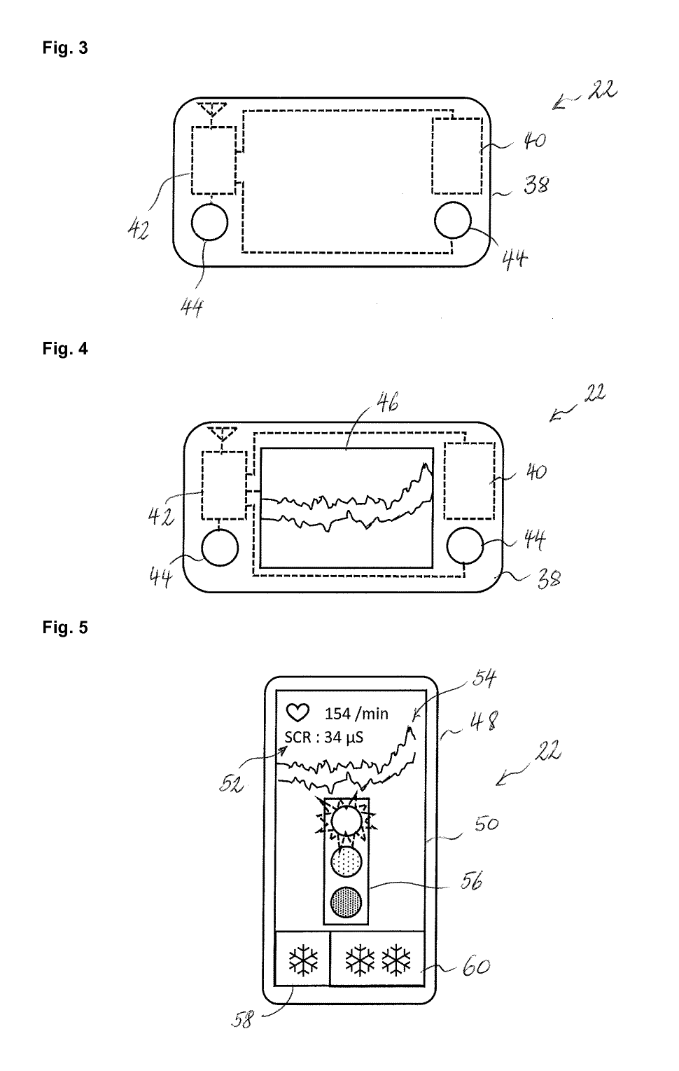

The monitor unit transmits the computed stress level (and, possibly, the biosignals) to the mobile terminals 22 held by the audience members. Embodiments of the mobile terminals are shown in FIGS. 3 to 5.

FIG. 3 illustrates a mobile terminal 22 taking the form of a simple handheld console 38 comprising an electromechanical actuator 40, e.g. an ERM motor, an LRA or a piezo actuator, a microcontroller 42 (including or wired to an RF module) and buttons 44 allowing sending a social touch. The microcontroller 42 receives the stress level from the monitor unit and converts it into a control signal for the electromechanical actuator 40, so as to provide a haptic feedback in the form of a heartbeat-like vibration to the person holding the console 38. The microcontroller 42 is configured so as to adjust the frequency of the heartbeat-like vibration to the stress level that has been communicated by the monitor unit. For instance, the microcontroller 42 may be configured to map the communicated stress level to one out of plural heartbeat-like vibration patterns. The heartbeat-like vibration patterns could e.g. be three in number and comprise a vibration resembling a calm heartbeat (e.g. 50 to 70 beats per minute), a vibration resembling an moderately accelerated heartbeat (e.g. 90 to 110 beats per minute) and a vibration resembling an accelerated heartbeat (e.g. 130 to 150 beats per minute). The microcontroller 42 also monitors whether one (or both) of the buttons 44 have been pressed. In that event, the microcontroller sends a signal to the first appliance 24, causing the latter to activate the cold applicators 18. The buttons 44 could be configured as simple on-off switches or as force-sensing actuators capable of detecting the amount of force exerted on the button. The amount, the intensity and/or the duration of the cold stimulus applied by the cold applicators could depend on the duration of the push of the button(s), on the amount of force applied on the buttons (if measured), and/or on whether only one button or both buttons have been pushed.

The duration of the cooling stimulus can be very brief and range, e.g. between 3 and 5 s, but longer durations are not excluded. Another parameter that can be controlled is the intensity of the stimulus. In case of a Peltier cooler, the intensity of the stimulus can be controlled by the amplitude of the current pulse sent through the Peltier cooler. The control circuitry 20 of the cold applicators 18 are configured to prevent too long and/or too intense and/or too frequent cooling stimuli.

FIG. 4 illustrates a variant of the mobile terminal 22 of FIG. 3, from which it differs only in that it comprises a display screen 46 for graphically displaying the measured biosignals and/or the stress level derived thereof. In all other respects, the mobile terminal of FIG. 4 is identical to the mobile terminal of FIG. 3.

FIG. 5 is an illustration of a smart phone 48 taking the role of a mobile terminal 22 thanks to installation of a corresponding app or plugin (adding functionality to an already installed app, e.g. a social media app). The app or plugin uses the smart phone's touch screen 50 as an input and output device. Additionally, the smart phone's electromechanical actuator(s) may be used by the app or plugin, e.g. to produce a heartbeat like vibration as described with reference to FIG. 3. In the illustrated embodiment, the app or plugin displays the current values of the measured biosignals 52 (heartbeat and skin conductance response, SCR), curves 54 representing the biosignals over a past time period and a stress level indicator 56. The app or plugin also displays buttons 58, 60 for administering cold to the speaker. In the illustrated example, pressing button 58 results in administration of a cold stimulus of comparatively low intensity and/or duration, whereas pressing button 60 results in administration of a cold stimulus of comparatively high intensity and/or duration.

If a smart phone is used as a mobile terminal of the present invention's communication system, it is not necessary that the smart phone's holder is physically present in the audience. It may be noted that in case of a plugin, the communication channels and other features of the hosting app may be used for communication between the different components of the present system as well as for input and output.

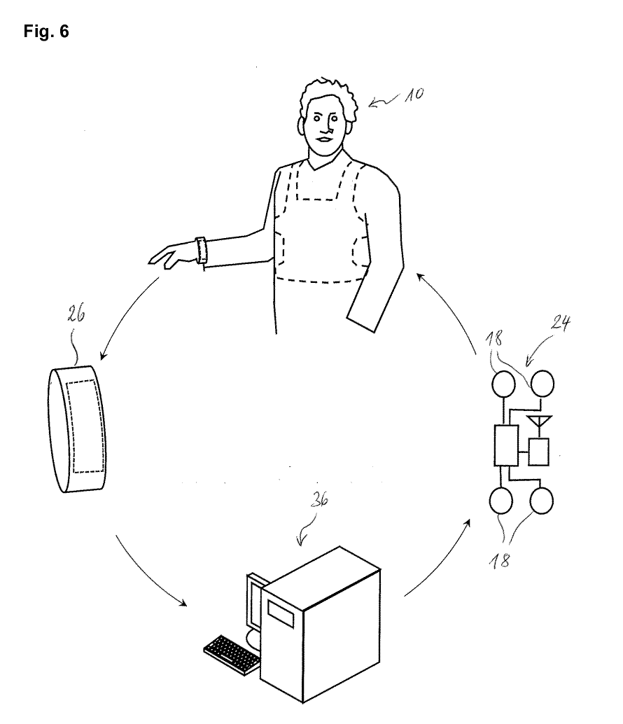

It will be appreciated that administration of cold stimuli could be automatized. FIG. 6 illustrates a communication system wherein the monitor unit (general purpose computer 36) autonomously recognises symptoms or elevated physiological stress and controls the cold applicators 18 of the first appliance 24 accordingly. In this mode of using the communication system, mobile terminals are unnecessary. Nevertheless, mobile terminals could be used complementarily. In this case, both the monitor unit and the mobile terminals could be at the origin of a cold stimulus.

It is worthwhile noting that FIGS. 2 and 6 merely show how the different components of the communication system influence one another on a high level. Physical communication paths may be different. For instance, messages from the second appliance (wristband 26) might have to transit via the first appliance before they are transmitted to the monitor unit, messages from the mobile terminals might have to transit via the monitor unit before they are transmitted to the first appliance, etc. Any suitable communication protocol(s) may be used for the communication between the different components of the communication system. Particularly preferred are, for instance, Bluetooth, WiFi, TCP/IP, etc.

It should also be noted that the monitor unit is not necessarily embodied separately from the mobile terminals or the first and second appliances. Specifically, the monitor unit could be implemented as a functional unit within the first appliance. Alternatively, the monitor unit could be implemented as a functional unit within one or more of the mobile terminals.

The communication system of FIGS. 1 to 6 serves as a tool for self-optimisation, measuring and allowing the reduction of stress during a public speaking task in real time. Previous monitoring systems were focused on recording human physiological signals; data processing and interpretation of the recorded data were carried out after the speech. By contrast, by using a system as disclosed herein, physiological signals (biosignals), such as the heart rate and electrodermal skin response may be tracked real-time and cooling stimuli for stress reduction may be applied real-time automatedly and/or by manual intervention of an audience member, a coach or any other person selected to provide support to the speaker.

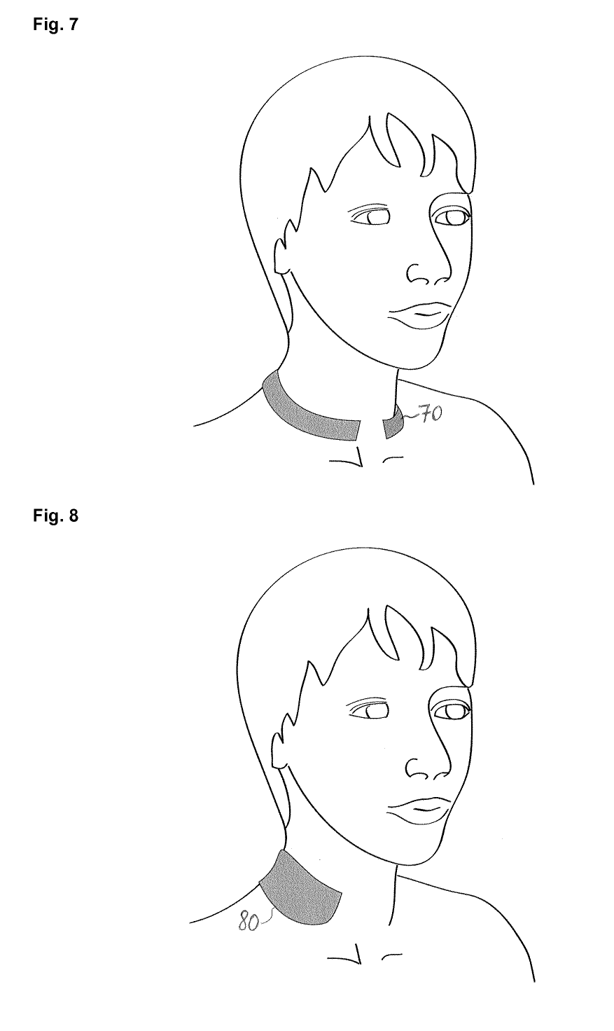

FIGS. 7 to 10 show illustrative embodiments of a wearable appliance employable in a communication system as described above or independently from it (as in FIG. 6), possibly in combination with a mobile device (e.g. smart phone, tablet, laptop computer in addition of or replacing the general purpose computer 36 of FIG. 6 as the monitoring unit) equipped with an app for configuring the wearable appliance.

Neck-ware versions of the wearable appliance can be categorized into a number of different form factors. In its basic version it is a “band” or neckwear item 70 that forms around the neck (see FIG. 7). The band, which is at least partly flexible or comprises one or more articulations to be put on or off, may be open or closed. The neckwear item 70 could be designed as a decorative necklace or disguised with a scarf, possibly one that attaches to the neckwear item 70.

The neckwear item is comprised of the cold applicators (e.g. Peltier micro modules, microspray cooling modules), a microcontroller, a wireless communication module, a heart rate sensor and a small battery. The microcontroller controls the cold applicators depending on the heart rate measured by the heart rate sensor and settings defined in the control software. Part or all of these settings may be modified via a user interface. The user interface could be accessible via the user's mobile phone running a dedicated app, a web-based form or any other suitable means. Preferably, certain settings are locked for editing by common users, especially safety-critical settings.

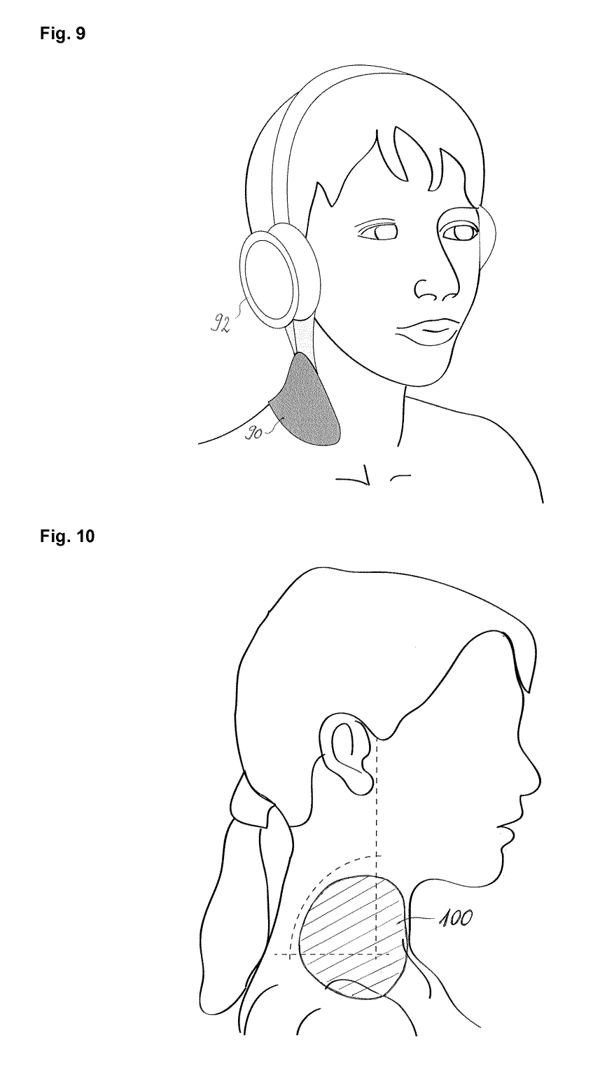

Other possible form factors of the (set of) wearable appliance(s) include one or more patches 80, 90 (see FIGS. 8 and 9) that can be attached to the neck with skin-friendly adhesive. The one or more patches may be made from powered smart materials that can create a cooling effect. The one or more patches 90 may be combined with a set of headphones 92 (see FIG. 9) or earphones. Alternatively, the one or more patches could be clipped on a shirt collar or be integrated into a specialized shirt, for example one that decreases sweating. They may also be combined with a halter that fits snug on the torso. The patch could then be integrated, e.g., into the collar of the halter.

Although embodiments capable of wirelessly interfacing with a remote (mobile) device may not need it, the wearable appliance or set of wireless appliances may comprise integrated therein a human-machine-interface (including e.g. a display screen, one or more buttons, a touchpad, etc.) allowing its configuration by the user.

A preferred location for application of the cold stimulus is on the side of the inferior portion of the neck as indicated in FIG. 10 by reference number 100. Some leeway may be given on every side of the indicated spot to accommodate for individual anatomical differences in the wearer. The spot 100 is preferably on the wearer's right-hand side where the neck connects to the torso. In this region, direct or indirect stimulation of specific nerves of the parasympathetic neural system (e.g. the vagus nerve or the trigeminal nerve) through cold stimuli is deemed particularly effective.

Preferred applications of the wearable appliance or the set of wearable appliances as a standalone unit, i.e. not in a collaborative context as described with reference to FIGS. 1 to 5, include the treatment of fear of public speaking and of fear of flying. When switched on, the wearable appliance or set of wearable appliances monitor the heart rate of the wearer and automatically administer them cold stimuli when an accelerated heart rate (indicative of acute stress) is detected. The cold pulses stimulate (directly or indirectly) the wearer's parasympathetic nervous system, which counteracts the sympathetic nervous system and thereby reduces the level of stress subjectively experienced by the wearer. Apart from applying cold pulses automatically, the wearable appliance or the set may give the wearer the possibility to manually administer himself cooling pulses.

The duration of the cooling stimulus can be very brief and range, e.g. between 3 and 5 s, but longer durations (e.g. in the range from 5 to 30 s) are not excluded. The durations of the cold pulses may be determined on-the-fly by the microcontroller (e.g. using artificial intelligence software that selects the most effective combination of duration and intensity to alleviate the wearer from stress). The user may be given the possibility to adjust the duration and/or the intensity of the stimuli via the user interface. In case of a Peltier cooler, the intensity of the stimulus can be controlled by the amplitude of the current pulse sent through the Peltier cooler. The control circuitry 20 of the cold applicators 18 are configured to prevent too long and/or too intense and/or too frequent cooling stimuli. If the cooling effect is achieved by spraying a cooling fluid onto a thermally conductive material that conveys the cold pulse on the skin (spraying the cooling fluid directly on the skin might be possible but is currently considered an inferior solution), amplitude and duration of the cold pulse depend on the choice of the cooling fluid, the duration of the spraying and the flow rate of the cooling fluid.

While specific embodiments and applications of the invention have been described herein in detail, those skilled in the art will appreciate that various modifications and alternatives to those details could be developed in light of the overall teachings of the disclosure. Accordingly, the particular arrangements disclosed are meant to be illustrative only and not limiting as to the scope of the invention, which is to be given the full breadth of the appended claims and any and all equivalents thereof.

1. A wearable appliance or a set of wearable appliances, arranged for being worn by a person on the person's body, the wearable appliance or the set comprising:
one or more cold applicators for applying cold to the person's body;
one or more biosignal sensors for measuring biosignals indicative of physiological stress experienced by the person; and
control circuitry connected to the one or more cold applicators and configured so as to activate the one or more cold applicators in response to detection, based upon the measured biosignals, that the person experiences elevated physiological stress.

2. The wearable appliance or set of wearable appliances as claimed in claim 1,
wherein the control circuitry comprises a monitor unit connected with the one or more biosignal sensors and configured for deducing the level of physiological stress from the biosignals and for monitoring the level of physiological stress or wherein the control circuitry is configured for being connected with such a monitor unit;
and wherein the control circuitry is configured for controlling the one or more cold applicators based upon the level of physiological stress deduced by the monitor unit.

3. The wearable appliance or set of wearable appliances as claimed in claim 1, wherein the one or more biosignal sensors comprise at least one of a heart rate sensor and an electrodermal activity sensor.

4. The wearable appliance or set of wearable appliances as claimed in claim 1, wherein the one or more cold applicators are arranged in such a way as to be located on the wearer's neck or on the superior portion of the torso where the torso connects to the neck.

5. The wearable appliance or set of wearable appliances as claimed in claim 4, comprising a neckwear item, wherein part of or all the cold applicators are located.

6. The wearable appliance or set of wearable appliances as claimed in claim 1, comprising one or more patches wherein part of or all the cold applicators are located, the patches being attachable to at least one of the wearer's skin, clothing and accessories worn on the body.

7. A communication system comprising:
a wearable appliance or a set of wearable appliances arranged for being worn by a first person on the first person's body, the wearable appliance or the set comprising one or more cold applicators for applying cold to the first person's body and one or more biosignal sensors for measuring biosignals indicative of physiological stress experienced by the first person;
a monitor unit configured for monitoring the level of physiological stress experienced by the first person, the level of physiological stress being deduced from the measured biosignals; and
one or more mobile terminals comprising each a human interface device with one or more output devices for indicating the level of physiological stress to a second person holding the mobile terminal and with one or more input devices for receiving input from the second person;
wherein the communication system is configured to translate input received by the one or more mobile terminals from one or more second persons into a control signal provided to the one or more cold applicators.

8. The communication system as claimed in claim 7, wherein the wearable appliance or at least one of the set of wearable appliances is integrated into a vest, a formfitting undergarment, a harness, a neckwear item or a halter with straps for being worn on the torso.

9. The communication system as claimed in claim 8, wherein the one or more cold applicators are arranged in the vest, formfitting undergarment, harness, neckwear item or halter.

10. The communication system as claimed in claim 9, wherein the one or more cold applicators are arranged in such a way as to be located on the first person's back or on the first person's neck or on the superior portion of the torso where the torso connects to the neck when the first person wears the vest, formfitting undergarment, harness, neckwear item or halter.

11. The communication system as claimed in claim 10, wherein the one or more cold applicators are at least two cold applicators arranged in such a way as to be located symmetrically on either side of the first person's spine or neck.

12. The communication system as claimed in claim 8, comprising a set of wearable appliances, wherein at least one of the set of wearable appliances is integrated into a wristband, a chest band, a neckwear item or a patch attachable to at least one of the first person's skin, clothing and accessories worn on the body.

13. The communication system as claimed in claim 12, wherein the at least one wearable appliance integrated into the wristband, chest band, neckwear item or patch comprises at least one of the one or more biosignal sensors.

14. The communication system as claimed in claim 7, wherein the monitor unit is integrated into the wearable appliance or into one of the set of wearable appliances.

15. The communication system as claimed in claim 7, wherein the monitor unit is integrated into a mobile terminal of the one or more mobile terminals.

16. The communication system as claimed in claim 7, wherein the monitor unit is separate both from the wearable appliance or the set of wearable appliances and from the mobile terminal.

17. The communication system as claimed in claim 7, wherein the one or more biosignal sensors comprise at least one of a heart rate sensor and an electrodermal activity sensor.

18. The communication system as claimed in claim 7, wherein the one or more cold applicators are at least two cold applicators and wherein the one or more mobile terminals are at least two mobile terminals, the mobile terminals being configured to send control signals to different cold applicators.

19. The communication system as claimed in claim 7, wherein the wearable appliance or the set of wearable appliances, the monitor unit and the one or more mobile terminals are configured to communicate by a wireless network.

20. The communication system as claimed in claim 7, wherein the output device of the mobile terminals include electromechanical actuators for haptically indicating the level of physiological stress.
